(19) United States
(12) Patent Application Publication
(10) Pub. No.: US 2007/0181119 A1
(43) Pub. Date: Aug. 9, 2007

(54) Inhaler Device

(76) Inventors: Robert E. Weinstein, Boston, MA (US); Allan M. Weinstein, Potomac, MD (US)

Correspondence Address: MESMER & DELEAULT, PLLC, 41 BROOK STREET, MANCHESTER, NH, 03104, US

(21) Appl. No.: 11/307,449

(22) Filed: Feb. 8, 2006

(51) Int. Cl. A61M 11/00; A61M 15/00; B65D 83/06

(52) U.S. Cl. 128/200.14; 128/200.23; 128/203.15

(57) ABSTRACT

An inhaler device to treat obstructive lung disease has an immediate acting bronchodilator medication, a dispenser housing for dispensing the immediate acting bronchodilator medication, and indicia on the dispenser housing to convey to a user that the inhaler device contains inhaler medication for for acute relief of obstructive lung disease symptoms.

### BACKGROUND OF THE INVENTION

1. Field of the Invention

The present invention relates to a device and method to reduce error in distinguishing topical inhaled agents used to treat respiratory tract disorders.

2. Description of the Prior Art

Individuals with obstructive pulmonary diseases are often required to utilize multiple medications, some of which are self-administered by inhalation. In the case of asthma, a disease characterized by shortness of breath, wheezing, cough, and excess sputum production, affected individuals often are required to use more than one inhaled medication. Asthmatic treatment typically requires the affected individual to use one inhaled medication regularly to control inflammation of the airway, and another inhaled medication to dilate the bronchial tree when symptoms occur that require immediate relief. This might be referred to as a regimen of “controller” and “rescue” medication. Medications for “controller” use act over many hours to days and include inhaled corticosteroids, examples being fluticasone, flunisolide, and triamcinolone, and long- acting bronchodilators, examples being salmeterol and formoterol. Short-acting beta-adrenergic agonists rapidly dilate the bronchial tree and their effects typically dissipate within a few hours. Examples of these inhaled medications that are used as “rescue” medication include albuterol, pirbuterol, and metaproterenol.

Rescue inhaler devices in current use have no structural characteristic to distinguish them from controller medication devices. The configurations of many of metered dose inhaler devices for rescue medication are the same or very similar to those of controller medication. For example, present brands of albuterol in metered dose inhalers include Proventil® and Proventil® HFA, manufactured by Schering, Ventolin® HFA manufactured by GlaxoSmithKline, and those manufactured by other pharmaceutical manufacturers that include Andryx, Armstrong, Ivax, and Warrick. None of these have any physical indication to convey to the user/patient that they are for immediate rescue use. Neither is any one of these products significantly distinguishable from controller medications on the market.

Examples of presently marketed metered dose controller inhalers that are similar in appearance to rescue medications are Aerobid® Inhaler containing flunisolide, manufactured by Forest, and Flovent® Inhalation Aerosol containing fluticasone proprionate, manufactured by GlaxoSmithKline. Were an individual to be prescribed GlaxoSmithKline's Flovent® as a ¢controller∞ and the same manufacturer's Ventolin® HFA for “rescue,” the two would be distinguishable only by name and color. Flovent® (a controller medication) is brick colored and Ventolin® HFA (a rescue medication) is blue. There is nothing about either the names or the colors that would suggest to a user which was for rescue, or which was for control. One might instinctively, and incorrectly, assume that the inhaler with the calming blue-gray color is the controller. Confusion such as this would be averted if rescue medications were clearly identified.

Therefore, what is needed is a rescue inhaler that has indicia to clearly distinguish it from controller medication and to convey to a user that it is to be used when required for immediate relief of symptoms.

### SUMMARY OF THE INVENTION

Despite an increasing understanding of obstructive lung diseases such as asthma and the availability of better medications, asthma morbidity and mortality has not improved. Failure by the patient/user to comply with treatment is an important underlying cause, and successful treatment requires the patient/user to understand which of the medications are required and their proper usage. The likelihood of inappropriate treatment and error increase when treatments are confusing and self-administered by the patient/user.

Confusion with treatment may occur when individuals are required to use a multiplicity of agents for treatment. In the case of multiple inhalers, individuals are known to confuse one with another. The inventors are allergists that have recognized that, despite careful instruction, it is not uncommon for patients to make errors and confuse controller and rescue medications. When asked “which inhaler do you use when you get into trouble?,” some patients incorrectly point to the controller inhaler. In some instances, this may be attributable to forgetfulness, or mistaking one for another, or an interval in which the medication was not used for a period and then resumed. In some instances, the confusion is heightened when the patient was prescribed one brand of medication, and a substitution made at the pharmacy to fill an equivalent brand or brands that have different appearance.

An object of the present invention is to correct a source of error by providing a user with a rescue inhaler that is clearly distinguishable and conveys its role to a user. It is another object of the present invention to provide a rescue inhaler having indicia that clearly distinguishes the rescue medication-containing inhaler from the controller medication-containing inhaler.

It is an object of the present invention to provide these and other objectives in an inhaler device having an inhaler dispenser housing with an indicia that conveys use for emergency relief from a bronchial attack and an aerosol canister that contains an immediate acting bronchodilator medication, also known as a rescue medication. The indicia may be a word or words, a symbol, a shape, or any combination that signifies emergency use. The indicia may also be integrated into the shape of the dispenser housing where the structure of the dispenser housing conveys the notion that the contents are for emergency use.

In one embodiment of the present invention, words are used to convey such a notion. Examples of wording to convey such an impression include, but are not limited to, “rescue,” “emergency use,” “fast-acting,” “for worsening symptoms,” “for shortness of breath,” “relief medication,” and the like.

In another embodiment of the present invention, color may be used alone or in combination with words, symbols, and shapes. An example of color is “fire engine” red.

In another embodiment, symbols are used to convey the impression that the contents of the inhaler device are for emergency use. Examples of symbols that may be used to convey a similar impression are a fire hose, an open bronchial tree, a life (rescue) ring, and the like.

In another embodiment, a shape is used to provide the notice function. An example of a shape includes a fire hydrant shape. Other shapes that denote “emergency use” and that are easily recognized by a user may be used, etc.

It is important that the dispenser housing of the inhaler device is configured so that upon seeing the inhaler device, the inhaler device conveys to a user/patient and the user/patient easily recognizes that the inhaler device contains “rescue” medication for the immediate relief of obstructive lung disease symptoms. In this way, user compliance with the treatment prescribed by the user's caregiver for the user's obstructive lung disease is improved.

### BRIEF DESCRIPTION OF THE DRAWINGS

FIG. 1 illustrates one embodiment of the present invention showing an inhaler device for rescue medication having indicia to identify the inhaler device as rescue medication.

FIG. 2 illustrates another embodiment of the present invention showing an inhaler device for rescue medication having fire hose indicia to identify the inhaler device as rescue medication.

FIG. 3 illustrates another embodiment of the present invention showing an inhaler device for rescue medication having lung indicia to identify the inhaler device as rescue medication.

FIG. 4 illustrates another embodiment of the present invention showing an inhaler device for rescue medication having a rescue ring indicia for identifying the inhaler device as rescue medication.

FIG. 5 illustrates another embodiment of the present invention showing an inhaler device for rescue medication having indicia that identifies the device as having emergency use medication.

FIG. 6 illustrates another embodiment of the present invention showing an inhaler device for rescue medication having a fire hydrant structure.

### DETAILED DESCRIPTION OF THE PREFERRED EMBODIMENTS

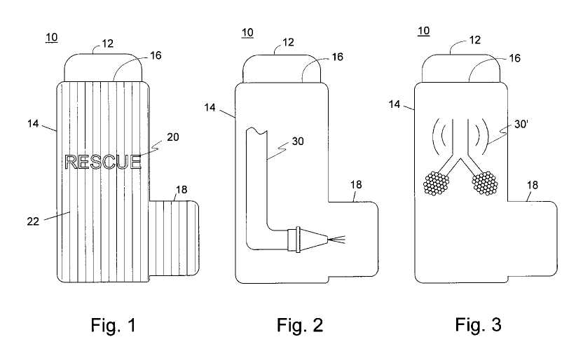

The preferred embodiment(s) of the present invention is illustrated in FIGS. 1-6. FIG. 1 depicts a side view of a metered-dose inhaler device 10 of the present invention. The inhaler device 10 includes an aerosol canister 12 and a dispenser housing 14. Dispenser Housing 14 has an aerosol canister recess 16 and a mouthpiece 18. Aerosol canister 12 contains an immediate acting bronchodilator medication, also known as a rescue medication. Aerosol canister 12 is fitted into dispenser housing 14 so that when actuated, aerosol canister 12 releases an aerosol spray through mouthpiece 18. Aerosol canister 12 is activated by the user pressing the canister 12 downward into housing 14. Dispenser housing 14 of inhaler device 10 has an indicia 20 with the wording “RESCUE” to convey to the user that the device is for immediate relief of symptoms. Optionally, dispenser housing 14 may also be colored a fire engine red as indicated by reference number 22. Other wording to convey similar meaning might be “emergency use,” “fast-acting,” “for worsening symptoms,” “for shortness of breath,“relief medication,” and the like.

FIG. 2 illustrates another embodiment of inhaler device 10. Inhaler device 10 is similar to the one depicted in FIG.1 but rather than a word indicia 20, housing 14 of inhaler device 10 contains an indicia 30 that depicts a shape associated with emergency use. In this arrangement, indicia 30 depicts a fire hose shape to indicate use for the immediate relief of symptoms.

FIG. 3 illustrates still another embodiment of inhaler device 10. Like the embodiment in FIG. 2, inhaler device 10 includes an indicia 30′. Indicia 30′ depicts an “open bronchial tree” to also indicate use for the immediate relief of symptoms and to convey to the user/patient that the medication contained in inhaler device 10 is used to “open” the bronchial network to facilitate breathing. Other indicia, such as a bright red color to denote emergency, a particular design or emblem, or a structural or topographic feature might also be used to immediately convey to the user that inhaler device 10 contains rescue medication as opposed to controller medication.

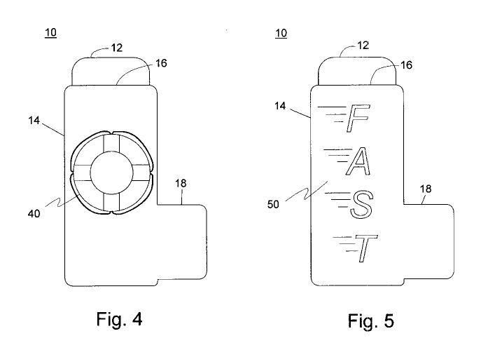

Turning now to FIG. 4, there is illustrated another embodiment of the present invention. Inhaler device 10 includes a dispenser housing 14 similar to the previously discussed housings 14 and includes an indicia 40. Indicia 40 has the shape of a rescue ring used in water rescue. Indicia 40 conveys that the aerosol canister 12 contains rescue medication for immediate relief of symptoms.

FIG. 5 illustrates another embodiment of the present invention. Inhaler device 10 includes a dispenser housing 14 with an indicia 50. Indicia 50 contains both a word or words and symbols. Indicia 50 has the word “FAST” with trailing lines (the symbols) on each letter emphasizing high speed. Indicia 50 conveys that inhaler device 10 contains rescue medication by implying that the medication will provide fast-acting relief of symptoms.

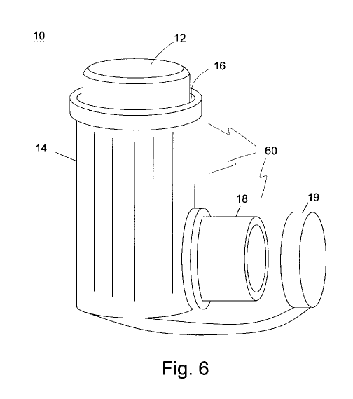

Turning now to FIG. 6, there is illustrated another embodiment of the present invention. The inhaler device 10 has a dispenser housing 14 with indicia 60. Indicia 60, however, are incorporated into the structural shape of dispenser housing 14. Dispenser housing 40 is preferably molded into the shape of a fire hydrant. Dispenser housing 40 may optionally include a “fire hydrant” cap 19 that fits over the end of mouthpiece 18. The “fire hydrant” shape conveys to the user/patient that a rescuing liquid/medication is contained within for immediate relief of symptoms.

The basic shape of inhaler device 10 in the Figures is meant to illustrate typical metered-dose inhalers that aerosolize medication in liquid form. It is within the scope of the present invention, however, to similarly provide indicia for other aerosolized rescue medications that might be employed by patients. Rescue medication in aerosolized powder form is one example. It is anticipated that manufacturers might manufacture separate indicia that can be applied to the inhaler device. It is considered most preferable, however, for the manufacturer to incorporate the indicia with the inhaler device prior to the user receiving it. Alternatively (though less preferred), an expert such as a pharmacist or other caregiver can apply the indicia to the dispenser housing 14 before the user receives it. However, it is not considered desirable to rely on the user to apply the indicia, due to the greater potential for error.

Although the preferred embodiments of the present invention have been described herein, the above description is merely illustrative. Further modification of the invention herein disclosed will occur to those skilled in the respective arts and all such modifications are deemed to be within the scope of the invention as defined by the appended claims.

What is claimed is:

1. An inhaler device containing inhaler medication to treat obstructive lung disease, said inhaler device comprising:
an immediate acting bronchodilator medication;
a dispenser housing for dispensing said immediate acting bronchodilator medication; and
indicia on said dispenser housing to convey to a user that said dispenser housing contains inhaler medication for acute relief of obstructive lung disease symptoms.

2. The inhaler device of claim 1 wherein said indicia is large enough to visibly convey to and be easily recognized by a user that said inhaler device contains inhaler medication for acute relief of obstructive lung disease symptoms.

3. The inhaler device of claim 1 wherein said indicia is selected from the group consisting of a color, wording, a figure, a shape, and a topographic feature wherein each item in said group conveys to the user that said inhaler medication contains rescue or fast-acting medication.

4. The inhaler device of claim 3 wherein said color is red.

5. The inhaler device of claim 1 wherein said dispenser housing has a shape that conveys to and is easily recognized by a user that said inhaler device contains inhaler medication for acute relief of obstructive lung disease symptoms.

6. The inhaler device of claim 1 wherein said immediate acting bronchodilator medication is albuterol.

7. The inhaler device of claim 1 wherein said inhaler device is a metered-dose inhaler.

8. A method of treating obstructive lung disease comprising the step of:
providing an immediate acting bronchodilator medication in an inhaler device for a user wherein said inhaler device contains rescue indicia to convey to a user that said inhaler device is used for acute relief of obstructive lung disease symptoms.

9. The method of claim 8 further comprising forming a dispenser housing of said inhaler device wherein said dispenser housing has one or more indicia selected from the group consisting of a color, wording, a figure, a shape, and a topographic feature wherein each item in said group is easily recognized by the user that said inhaler device contains rescue or fast-acting medication.

10. An inhaler device for treating obstructive lung disease with an immediate acting bronchodilating medication, the improvement comprising means for visibly conveying to and being easily recognized by a user that said inhaler device contains inhaler medication for immediate relief of obstructive lung disease symptoms.

11. The inhaler device of claim 10 wherein said means are indicia.

12. The inhaler device of claim 11 wherein said indicia is selected from the group consisting of a color, wording, a figure, a shape, and a topographic feature wherein each item in said group conveys to the user that said inhaler medication contains rescue or fast acting medication.

13. The inhaler device of claim 12 wherein said color is red.

14. The inhaler device of claim 12 wherein said indicia is selected from the group consisting of a fire hose, a life ring, an illustration of an open bronchial tree, and a combination of wording and a shape.

15. The inhaler device of claim 12 wherein said topographic feature is structured as a fire hydrant.
